(19) United States
(12) Patent Application Publication
(10) Pub. No.: US 2026/0166259 A1
(43) Pub. Date: Jun. 18, 2026

(54) SYSTEM AND METHOD FOR PROVIDING PERSONALIZED STIMULI BASED ON BIO-SIGNALS

(71) Applicant: NeuroTx Co., Ltd., Seoul (KR)

(72) Inventors: Dong-Joo KIM, Seoul (KR); Goeun KIM, Seoul (KR); Donghyeok JO, Seoul (KR)

(73) Assignee: NeuroTx Co., Ltd., Seoul (KR)

(21) Appl. No.: 19/003,423

(22) Filed: Dec. 27, 2024

(30) Foreign Application Priority Data
Dec. 12, 2024 (KR) 10-2024-0184337

(51) Int. Cl. A61M 21/02; A61M 21/00

(52) U.S. Cl. CPC A61M 21/02; A61M 2021/0027; A61M 2021/005; A61M 2205/3553; A61M 2205/3561; A61M 2205/3584; A61M 2230/06; A61M 2230/10; A61M 2230/63

(57) ABSTRACT

According to an example embodiment, the invention relates to a personalized stimulus delivery system and method, which collects real-time biometric signal data from users, analyzes it, and provides optimized stimuli. More specifically, it involves a technology that collects ECG (electrocardiogram), EEG (electroencephalogram), SPO₂ (blood oxygen saturation), Actigraphy (physical activity), and environmental noise data, then evaluates the user's stress level, sleep quality, and physical fatigue based on this data. According to the analysis results, personalized stimuli such as binaural beats, ASMR, and frequency-based stimuli are provided. The example embodiment performs data preprocessing and filtering to enhance the accuracy and stability of the biometric signal data, and optimizes and delivers stimuli that reflect the user's state and preferences through artificial intelligence algorithms. Additionally, it includes a system that evaluates the effectiveness of the stimuli through real-time feedback and continuously improves the stimulus settings. The example embodiment is designed with a user-centered focus on health management and well-being enhancement, aiming to provide various benefits such as stress relief, improved sleep quality, and increased concentration.

### CROSS-REFERENCE TO RELATED APPLICATION

This application claims priority from and the benefit of Korean Patent Application No. 10-2024-0184337 filed on Dec. 12, 2024, in the Korean Intellectual Property Office, the disclosure of which is incorporated herein by reference.

### BACKGROUND OF THE DISCLOSURE

### Field of the Disclosure

Example embodiments relate to a system and method for providing personalized stimuli by receiving and analyzing a user's biometric signal data in real time. More specifically, the embodiments pertain to a technology for evaluating stress levels and psychological stability based on various biometric data such as Heart Rate Variability (HRV), Electroencephalogram (EEG), and Blood Oxygen Saturation (SpO₂), and determining optimal stimuli, such as binaural beats and ASMR content, to enhance the user's well-being.

### Description of the Related Art

Advancements in wearable devices and the integration of healthcare technologies have enabled the design of user-customized health management systems, drawing significant attention to stimuli delivery technologies based on biometric signals.

In particular, technologies aimed at receiving a user's biometric signal data in real time to induce effects such as stress relief, improved sleep quality, and enhanced concentration are rapidly evolving. Representative approaches include receiving data through wearable devices such as ECG (Electrocardiography), EEG (Electroencephalogram), SpO₂ (Blood Oxygen Saturation), Actigraphy (Physical Activity), and environmental noise, and analyzing the data using AI-based algorithms.

Wearable devices receive biometric signals in real time and transmit the data to a server for analysis, evaluating stress levels, sleep states, and physical fatigue. ECG is utilized to analyze heart rate variability, EEG to classify sleep stages, and SpO₂ to assess respiratory conditions. Actigraphy evaluates rest and sleep quality based on movement patterns and activity levels, while environmental noise data identifies factors that disrupt sleep.

Machine learning algorithms process this data to generate and optimize user-customized stimuli, providing the most effective stimuli type through personalized models. Users are offered real-time feedback and data visualization features, allowing them to intuitively monitor and track their health status based on the received data.

However, biometric signal data is highly sensitive to external environmental factors, making it prone to noise, which may lower the reliability of the analysis results. Insufficient data preprocessing may lead to inappropriate stimuli being delivered. Moreover, many systems fail to adequately reflect individual user characteristics and preferences, resulting in uniform stimuli that may not have the same effect on all users.

Synchronization issues may arise during the real-time processing of biometric signals and the delivery of stimuli, leading to mismatches between the timing of the stimuli and the user's state. Non-intuitive technical designs may degrade user experience, and existing systems often lack the ability to quantitatively evaluate the effects of stimuli or to continuously optimize stimuli design post-delivery.

### SUMMARY

An objective of example embodiments is to provide a system capable of effectively evaluating stress levels, sleep stages, and fatigue levels by receiving and analyzing a user's real-time biometric signal data.

An objective of example embodiments is to determine personalized stimuli based on biometric signal data and deliver them at the optimal timing to improve the user's condition.

An objective of example embodiments is to ensure the accuracy and reliability of biometric signal data by enhancing secure and reliable communication for data transmission and processing.

An objective of example embodiments is to provide a customized health management system that is easy to use for anyone by designing a simple and intuitive user interface.

An objective of example embodiments is to analyze the effects of delivered stimuli and continuously optimize personalized stimuli settings through iterative learning of the data.

According to an example embodiment, there is provided a system for providing personalized stimuli, wherein the system comprises: a data receiving unit configured to receive a user's biometric signal data in real time, a data preprocessing unit configured to preprocess the received biometric signal data by removing noise and normalizing the data, a data analyzing unit configured to analyze the preprocessed biometric signal data to calculate an HRV (Heart Rate Variability) index, a stimulation determining unit configured to determine an optimal stimulation by calculating at least one rate of change of the HRV index for resting intervals based on the calculated HRV index, and a stimulation providing unit configured to provide the determined optimal stimulation for a predetermined period of time.

According to an example embodiment, there is provided the system, wherein the data receiving unit is configured to stack data on a local device in predetermined time units through short-range wireless communication and to upload the data to a cloud storage.

According to an example embodiment, there is provided the system, wherein the data receiving unit is configured to store biometric signal data in a structured JSON (JavaScript Object Notation) format along with metadata including user ID, experiment ID, timestamp, and data file index.

According to an example embodiment, there is provided the system, wherein the data preprocessing unit is configured to perform noise removal and normalization on the received biometric signal data and to segment voice data using a library.

According to an example embodiment, there is provided the system, wherein the data analyzing unit is configured to calculate WASO (Wake After Sleep Onset), total sleep time, major rest periods, and sleep onset latency based on actigraphy data.

According to an example embodiment, there is provided the system, wherein the data analyzing unit is configured to calculate a user-specific sleep score by integrating HRV indicators, EEG sleep stage distributions, and actigraphy indicators based on the user's sleep data.

According to an example embodiment, there is provided the system, wherein the data analyzing unit is configured to classify sleep stages from the received biometric signal data using a pre-trained artificial intelligence model.

According to an example embodiment, there is provided the system, wherein the stimulation determining unit is configured to calculate the rate of change in the HRV index for each resting interval relative to a baseline index and to compute a comprehensive score by applying weights to each index.

According to an example embodiment, there is provided the system, wherein the stimulation determining unit is configured to select the stimulation setting with the highest comprehensive score based on the optimal rate of change in indicators for each stimulation setting option.

According to an example embodiment, there is provided the system, wherein the stimulation providing unit is configured to provide stimulation including binaural beats for the duration of the predetermined stimulation setting.

According to an example embodiment, there is provided a method of operating a system for providing personalized stimuli, wherein the method comprises, receiving a user's biometric signal data in real time, preprocessing the received biometric signal data by removing noise and normalizing the data, analyzing the preprocessed data to calculate an HRV (Heart Rate Variability) index, determining an optimal stimulation by calculating a rate of change of the HRV index for each resting interval based on the calculated HRV index, and providing the determined optimal stimulation for a predetermined period of time.

According to an example embodiment, there is provided the method, wherein the step of receiving the biometric signal data in real time comprises, stacking the biometric signal data on a local device in predetermined time units via short-range wireless communication and uploading the data to cloud storage.

According to an example embodiment, there is provided the method, wherein the step of determining an optimal stimulation by calculating a rate of change of the HRV index for each resting interval based on the calculated HRV index comprises, calculating the rate of change of the HRV index for each resting interval relative to a baseline index and calculating a comprehensive score by applying weights to the respective indices.

According to an example embodiment, there is provided the method, wherein the step of determining an optimal stimulation by calculating a rate of change of the HRV index for each resting interval based on the calculated HRV index comprises, selecting the stimulation setting with the highest comprehensive score based on the optimal rate of change of the index for each stimulation setting option.

According to an example embodiment, there is provided a non-transitory computer-readable storage medium, wherein the medium has programmable instructions stored therein that, when executed by one or more hardware processors, cause the one or more hardware processors to, receive a user's biometric signal data in real time, preprocess the received biometric signal data to remove noise and normalize the data, analyze the preprocessed data to calculate an HRV (Heart Rate Variability) index, determine an optimal stimulation by calculating a rate of change of the HRV index for each resting interval based on the calculated HRV index, and provide the determined optimal stimulation for a predetermined period of time.

According to an example embodiment, a user may monitor biometric signal data in real time and intuitively understand their condition.

According to an example embodiment, personalized stimuli based on biometric signal indicators such as HRV may be provided to alleviate stress and enhance psychological stability.

According to an example embodiment, noise removal and data normalization may improve the accuracy of biometric signal data analysis.

According to an example embodiment, by learning from historical data, stimuli that reflect the user's preferences and condition may be provided, contributing to continuous improvement of the user experience.

According to an example embodiment, personalized stimuli settings may improve sleep quality and support the development of regular sleep patterns.

### BRIEF DESCRIPTION OF THE FIGURES

Embodiments will be described in more detail with regard to the figures, wherein like reference numerals refer to like parts throughout the various figures unless otherwise specified, and wherein:

FIG. 1 is a diagram illustrating a system for providing personalized stimuli according to an example embodiment.

FIG. 2 is a diagram illustrating the basic structure of the application according to an example embodiment.

FIG. 3 is a diagram illustrating the application-server communication method.

FIG. 4 is a diagram illustrating the application-server communication method for biometric signal measurement.

FIG. 5 is a diagram illustrating the algorithm for personalized stimuli settings.

FIG. 6 is a diagram illustrating sleep analysis through batch scheduling.

FIG. 7 is a comprehensive diagram illustrating the content of the WillSleep app.

FIG. 8 is a diagram illustrating the personalized mode for stimuli in the app service.

FIG. 9 is a diagram illustrating the pre-set mode in the app service.

FIG. 10 is a diagram illustrating ASMR content that users may play in the app service.

FIG. 11 is a diagram illustrating the screen with binaural beats in the app service.

FIG. 12 is a diagram illustrating the self-assessment feature in the app service.

FIG. 13 is a diagram illustrating the sleep quality scale (PSQI) in the app service.

FIG. 14 is a diagram illustrating the insomnia severity index (ISI) in the app service.

FIG. 15 is a diagram illustrating the anxiety index (BAI) in the app service.

FIG. 16 is a diagram illustrating the depression index (BDI) in the app service.

FIG. 17 is a diagram illustrating bio-signals in the app service.

FIG. 18 is a diagram illustrating sleep analysis in the app service.

FIG. 19 is a diagram illustrating the sleep calendar in the app service.

FIG. 20 is a diagram illustrating the alarm feature in the app service.

FIG. 21 is a diagram illustrating the newsletter feature in the app service.

FIG. 22 is a diagram illustrating the method of operation for the system for providing personalized stimuli according to an example embodiment.

### DETAILED DESCRIPTION OF THE DISCLOSURE

Hereinafter, the specific structural or functional descriptions of embodiments according to the concept of the present invention disclosed herein are merely illustrative for the purpose of explaining the embodiments of the concept of the present invention. The embodiments of the concept of the present invention may be implemented in various forms and are not limited to the embodiments described herein.

Embodiments according to the concept of the present invention may undergo various modifications and take various forms. Therefore, the embodiments are illustrated in the drawings and described in detail in this specification. However, these are not intended to limit the embodiments to the specific disclosed forms, and they include modifications, equivalents, or substitutes that fall within the spirit and scope of the present invention.

Terms such as “first” or “second” may be used to describe various components but should not limit the components by these terms. These terms are only used to distinguish one component from another. For example, within the scope of the concept of the present invention, a “first component” may also be referred to as a “second component,” and similarly, a “second component” may also be referred to as a “first component.”

When a component is referred to as being “connected to” or “coupled to” another component, it should be understood that the component may be directly connected or coupled to the other component, or there may be intervening components. In contrast, when a component is referred to as being “directly connected to” or “directly coupled to” another component, it should be understood that there are no intervening components. Similarly, expressions describing relationships between components, such as “between” and “directly between” or “adjacent to,” should be interpreted in the same way.

The terminology used herein is for the purpose of describing specific embodiments and is not intended to limit the present invention. Singular expressions include plural expressions unless the context clearly indicates otherwise. Terms such as “comprises” or “has” as used herein specify the presence of stated features, numbers, steps, operations, components, elements, or combinations thereof, but do not preclude the presence or addition of one or more other features, numbers, steps, operations, components, elements, or combinations thereof.

Unless otherwise defined, all terms used herein, including technical and scientific terms, have the same meaning as commonly understood by one of ordinary skill in the art to which the present invention belongs. Terms generally defined in commonly used dictionaries should be interpreted as having meanings consistent with the context of the relevant technology, and unless explicitly defined herein, should not be interpreted in an idealized or overly formal sense.

Hereinafter, embodiments will be described in detail with reference to the accompanying drawings. However, the scope of the patent application is not limited or restricted by these embodiments. Identical reference numerals in each drawing denote the same elements.

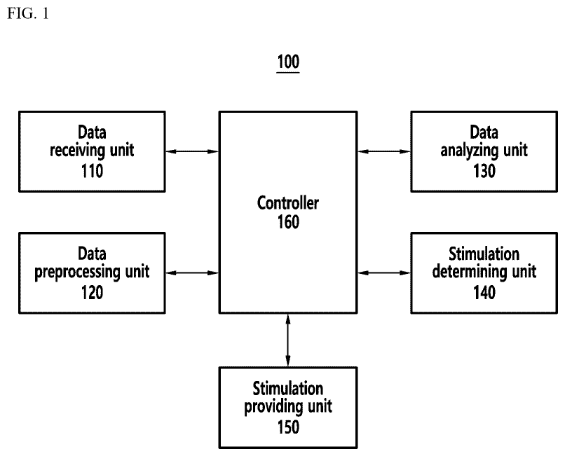

FIG. 1 illustrates the main components of the system for providing personalized stimuli 100 according to the present invention, detailing the data flow between components and their functional interconnectivity.

The system for providing personalized stimuli 100 according to an example embodiment is designed to analyze, process, and determine personalized stimuli based on biometric signal data received from users and provide them to improve sleep, alleviate stress, enhance concentration, and promote psychological stability.

The system for providing personalized stimuli 100 according to an example embodiment may include: a data receiving unit 110, a data preprocessing unit 120, a data analyzing unit 130, a stimulation determining unit 140, a stimulation providing unit 150, and a controller 160.

According to an example embodiment, the data receiving unit 110 receive a user's biometric signal data in real time. Specifically, it stacks the data on a local device in predetermined time units via short-range wireless communication and uploads it to cloud storage. Additionally, the data receiving unit 110, according to an example embodiment, stores the biometric signal data in a structured JSON (JavaScript Object Notation) format along with metadata including user ID, experiment ID, timestamp, and data file index.

According to an example embodiment, the data preprocessing unit 120 preprocesses the received biometric signal data by removing noise and normalizing the data. Specifically, the data preprocessing unit 120 performs noise removal and normalization on the received biometric signal data and segments voice data using a library.

According to an example embodiment, the data analyzing unit 130 analyzes the preprocessed data to calculate an HRV (Heart Rate Variability) index. Specifically, the data analyzing unit 130 calculates WASO (Wake After Sleep Onset), total sleep time, major rest periods, and sleep onset latency based on actigraphy data. The data analyzing unit 130 integrates HRV indicators, EEG sleep stage distributions, and actigraphy indicators based on the user's sleep data to calculate a user-specific sleep score. Additionally, the data analyzing unit uses a pre-trained artificial intelligence model to classify sleep stages from the received biometric signal data.

According to an example embodiment, the stimulation determining unit 140 determines the optimal stimulation by calculating the rate of change of the HRV (Heart Rate Variability) index for each resting interval based on the calculated HRV index.

Specifically, the stimulation determining unit 140 calculates the rate of change of the HRV index for each resting interval relative to a baseline index and computes a comprehensive score by applying weights to the respective indicators. Additionally, the stimulation determining unit 140 selects the stimulation setting with the highest comprehensive score based on the optimal rate of change of the HRV index for each stimulation setting option.

According to an example embodiment, the controller 160 integratively controls all components and manages data flow. Each component is organically interconnected, enabling real-time data processing and the provision of personalized stimuli to users.

According to an example embodiment, the data receiving unit 110 receives various biometric signal data from users in real time, providing the initial input data for the system. The received data includes heart rate (HR), heart rate variability (HRV), electroencephalogram (EEG), actigraphy data, and sound data.

Heart rate measures the intensity and regularity of cardiac activity, while HRV analyzes the variability in time intervals between heartbeats to assess the balance of the autonomic nervous system. EEG provides data for analyzing sleep stages, and actigraphy data records the user's activity levels and rest cycles based on physical movement. Sound data receives ambient noise to analyze its impact on the sleep environment.

According to an example embodiment, the data receiving unit 110 connects to devices such as smartwatches, ECG/EEG measurement devices, and sound sensors via BLE communication to receive data. The data is received in real time and stacked on a local device in 5-minute intervals to prevent data loss. The received data is then uploaded to a cloud platform, such as Cloud Storage, allowing for additional analysis on a central server.

The data uploaded to the cloud is stored in JSON format along with a data file index, including metadata such as user ID, experiment ID, and timestamp. Real-time data collection and cloud uploads ensure reliability and scalability, while unique file indexing is managed to prevent data loss.

The data preprocessing unit 120 refines the biometric signal data provided by the data receiving unit, converting it into an analyzable state.

In particular, the data preprocessing unit 120 enhances the quality of the data and improves the accuracy of analysis.

Noise removal involves refining ECG data using a library and eliminating background noise from sound data using a library.

Received data of various sizes and units are normalized to enable analysis and comparison, and sound data is segmented to structure it for time-based analysis. EEG data is normalized before being input into a classification model. The preprocessed data is transmitted to the data analyzing unit 130, ensuring it remains optimized for analysis.

The data analyzing unit 130 generates various indicators to assess the user's condition based on the preprocessed data. For HRV analysis, it calculates HRV metrics such as SDNN, RMSSD, and the LF/HF ratio to evaluate the state of the autonomic nervous system. In EEG-based sleep stage analysis, it utilizes the pre-trained SleepExperNet AI model to analyze EEG data and classify sleep stages into Wake, N1, N2, N3, and REM.

The actigraphy data analysis evaluates the user's physical movements to assess parameters such as Wake After Sleep Onset (WASO), total sleep time, and major rest periods. The analyzed results are stored in a user-specific database and forwarded to the stimulation determining unit.

The stimulation determining unit 140 determines the optimal stimulation settings to be provided to the user based on the results derived from the data analyzing unit 130. It calculates the rate of change in HRV metrics compared to baseline data, assigns weights to each metric, and computes a comprehensive score. The stimulation setting with the highest score is selected.

The stimulation providing unit 150 delivers the determined optimal stimulation for the set duration. Specifically, based on the settings selected by the stimulation determining unit 140, it provides the user with physical or auditory stimuli. The provided stimuli include binaural beats and are configured according to the user's selected duration (10 minutes, 20 minutes, 30 minutes). Additionally, it may be adjusted based on user preferences, including a mute option.

For example, the stimulation providing unit 150 delivers stimulation that includes binaural beats for the selected stimulation duration.

The controller 160 coordinates the data flow between all components of the system for providing personalized stimuli 100 according to an example embodiment and manages the overall operation of the system. It monitors data flow and system status, detects and recovers from errors, and orchestrates task timing and prioritization.

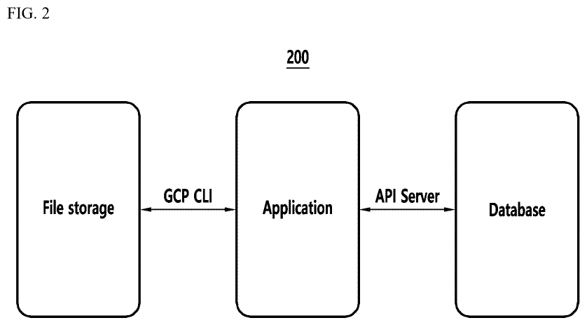

FIG. 2 illustrates the basic structure of the application according to an example embodiment.

In particular, it explains the detailed components of the application's basic structure 200, the data flow between components, the communication methods, and their functional roles. This application structure 200 is designed to efficiently collect, store, process, and analyze a user's biometric signal data and provide personalized stimuli.

The system for providing personalized stimuli 100, according to an example embodiment, consists of a client application, cloud storage, an API server, and a database. Each component is interconnected to process and store data in real time and provide user-specific feedback.

File Storage is a cloud-based storage system for large-scale storage of user biometric signal data, utilizing the Cloud Storage service provided by the Cloud Platform (GCP). File Storage receives and stores biometric data, enabling analysis and processing by the application and server. The biometric signal data includes various types of data such as heart rate, heart rate variability (HRV), electroencephalogram (EEG), actigraphy data, and sound data. The data is structured in JSON format to facilitate data processing and retrieval.

The received data is uploaded to the cloud from the application every 5 minutes using the GCP CLI (Command Line Interface) and stored with distinctions based on file indexing, including user ID, experiment ID, and timestamp.

Cloud-managed data allows users to access it anytime and anywhere, providing connectivity with various devices through a centralized data storage system.

The data stored in File Storage is saved in JSON format and is organized by user for efficient retrieval and processing.

The JSON file provides a structured format that enables efficient processing and retrieval of user data. The storage system ensures scalability and stability, making it suitable for handling large volumes of data. To prevent data loss, it uses multi-region backups and unique file indexing to manage data.

The application serves as the core client module responsible for the user interface (UI) and data processing, implemented using the Flutter framework. It receives data from devices such as smartwatches, ECG monitors, and EEG devices via BLE (Bluetooth Low Energy) communication. The received data is temporarily stored locally before being uploaded to File Storage using the GCP CLI.

The application presents real-time received biometric data in graphical or visual dashboard formats and intuitively displays analysis results and stimulation setting data to users. Based on personalized stimulation setting data, it provides binaural beats or user-defined auditory stimuli.

The application is implemented as a platform-independent solution using Flutter, enabling it to operate on both Android and iOS. It provides data feedback through user-customized settings (e.g., stimulation duration, stimulation intensity) and a graphical interface, offering real-time data visualization with platform independence and a responsive UI.

The API server connects the client application and the database, handling core business logic and data processing. It processes client requests (e.g., data retrieval, returning analysis results) through RESTful APIs, downloads data from File Storage, validates it, and performs analysis.

The analyzed data is stored in the database and delivered to the client when needed. Based on the analysis results, the system generates stimulation setting data tailored to the user's condition (e.g., stress level, sleep quality). The API server is implemented using the Spring Framework, providing flexibility and scalability.

According to an example embodiment, data encryption and authentication (e.g., JWT) between the server and client enhance security, supporting reliable data processing and communication.

The database serves as a central repository for storing user biometric signal data and analysis results. It is implemented using MySQL and operates in a Cloud SQL environment.

The database is structured and managed on a per-user basis, maintaining data integrity. It stores biometric signal data (e.g., HR, HRV, EEG indicators) and analysis results (e.g., WASO, sleep stages, stress indicators) and returns analysis results based on client requests.

The schema is designed based on user ID, timestamp, data type, and analysis results, ensuring data stability through an automated backup and recovery system.

The data flow between File Storage and the Application involves receiving data via BLE, locally stacking it, and uploading it to Cloud Storage using the GCP CLI. The Application communicates with the API Server through RESTful APIs, while the API Server interacts with the database using SQL queries.

File Storage and the API Server communicate by having the server download data from Cloud Storage, analyze it, and return the results.

This entire data flow utilizes GCP-based cloud storage and a MySQL database to ensure scalability and stability. The RESTful API and Spring Framework-based server architecture enable flexible responses to various requests.

According to an example embodiment, real-time data collection and analysis are supported using BLE communication and cloud infrastructure, while user information security is enhanced through data encryption and authentication systems.

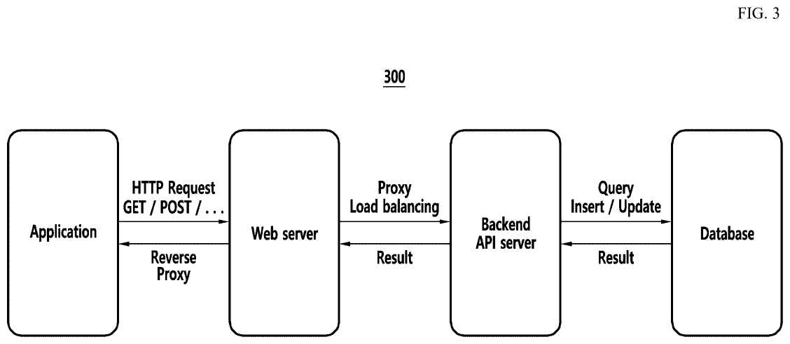

FIG. 3 specifically illustrates the communication method between the application and the server according to the present invention, describing an architecture consisting of a client (Application), web server (Web Server), backend API server (Backend API Server), and database (Database).

The structure in FIG. 3 explains the entire process of handling, storing, analyzing user request data, and returning responses. Each component is designed to improve data processing speed and accuracy while enhancing system stability.

The application serves as the client-side interface, enabling users to send requests to the server and receive responses.

This client is implemented using the Flutter framework, supporting multiple platforms (Android and iOS). The application generates HTTP requests for tasks such as data retrieval, analysis requests, and personalized stimulation settings requested by users, with the request body structured in JSON format.

Additionally, the application presents data or analysis results received from the server in a dashboard format, visualizing the data through graphs and charts for the user.

Biometric data is received from wearable devices using BLE (Bluetooth Low Energy) and transmitted to the server while maintaining the security of data requests and responses through the HTTPS protocol and JWT (JSON Web Token).

The server response data is transformed into user-specific information and displayed through the UI. For example, the user's HRV metric changes and stress analysis results are provided.

The web server acts as a middle layer between the client and the backend server, performing tasks such as request distribution, security processing, and data encryption. It forwards client requests to the backend API server while protecting the internal API server from external requests.

The web server distributes requests across multiple backend API servers, reducing the load on each server and optimizing response times. Additionally, it maintains balance among servers using load-balancing algorithms such as Round Robin and Least Connections.

The web server encrypts data transmission between the client and server using HTTPS, functions as a basic firewall, and blocks malicious requests. It routes client requests to the appropriate API server based on the URL path and HTTP method specified in the request.

The backend API server performs core business logic, processes client requests, and interacts with the database to store or retrieve data. The backend API server validates the client request data and verifies authentication and authorization through JWT tokens.

Based on the request, the backend API server analyzes biometric data or calculates personalized stimulation settings and returns the results to the client.

For example, the backend API server analyzes HRV data to calculate stress levels or evaluate sleep states.

The backend API server performs data insertion, retrieval, updating, and deletion using SQL queries, processes the data according to client requests, and returns it.

It handles requests forwarded by the web server, accesses the database to retrieve or update the required data, and converts the processed results into JSON format, which is returned to the client as the response body.

The database serves as a central repository for storing and managing user data and analysis results. It is designed based on MySQL and operates in a Cloud SQL environment.

The database stores user IDs, biometric signal data (HR, HRV, EEG), analysis results, and stimulation setting data. It quickly retrieves the required data according to client requests and returns it to the API server.

The database ensures data stability through regular backups and a recovery system. The database tables include user information, biometric signal data, analysis results, and log data, which are efficiently managed through normalization.

HTTP requests generated by the application are sent to the web server.

The request body is structured in JSON format and includes request data and authentication information. The web server analyzes client requests and forwards them to the appropriate API server, distributing the requests across multiple API servers using load balancing.

The API server sends SQL queries to the database to insert or retrieve data, and the database returns the processed results to the API server. The API server processes the results retrieved from the database and returns them to the web server, which then delivers the data to the client application. The delivered data is displayed in the user interface (UI).

This architecture provides scalability to handle a large number of user requests through multiple API servers and database clustering. It also maintains high reliability through the web server's load balancing and the database's backup system.

According to an example embodiment, data transmission security is enhanced using HTTPS and JWT authentication, and the internal network is protected with a reverse proxy. Additionally, the optimized data transmission speed between the client and server reduces request and response times.

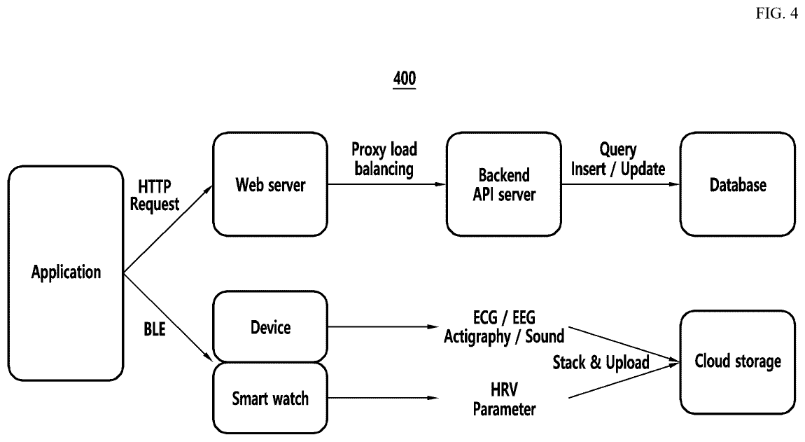

FIG. 4 is a diagram illustrating the communication method between the application and the server for biometric signal measurement. Specifically, FIG. 4 details the process of processing and transferring biometric signal measurement data among the application, server, and storage. The structure in FIG. 4 consists of an application, device, smart watch, web server, backend API server, database, and cloud storage. Each component performs roles such as data collection, storage, processing, analysis, and transmission.

The system according to the present invention is designed for real-time data processing and stability, offering data integration and personalized analysis results to users.

The application serves as a client-side interface where users may visualize biometric data, communicate with the server, and perform analysis requests. Developed using the Flutter framework, the application connects to devices and smartwatches via BLE (Bluetooth Low Energy).

The application receives biometric data in real time via BLE and transmits the received data to the server. It generates HTTP requests to send data to the server, perform analysis requests, and receive results. The application uploads data to Cloud Storage and stacks data at 5-minute intervals to prevent data loss. It presents analysis results received from the server to the user in the form of dashboards and graphs.

The application ensures the security of data requests and transmissions using JWT (JSON Web Token). It visualizes real-time biometric data through graphs and charts, providing personalized feedback to users based on the analysis results.

The device according to the present invention is hardware connected to the application via BLE, equipped with various sensors to measure and store biometric data. The device performs real-time data transmission and basic data preprocessing.

The device receives data through ECG (Electrocardiogram) to calculate HRV (Heart Rate Variability). It provides data for classifying sleep stages (Wake, N1, N2, N3, REM) based on EEG (Electroencephalogram) data. The device measures information such as physical activity and sleep cycles using actigraphy data.

Additionally, the device receives sound data to analyze environmental factors (e.g., noise) and the user's sleep state. The received data is stacked at regular intervals and uploaded to Cloud Storage.

The smartwatch measures the user's biometric data in real time and connects to the application via BLE.

The device plays a supplementary role in receiving wearer-centric data and transmitting it to the server.

The device according to the present invention receives key biometric data such as HR (Heart Rate), SPO₂ (Blood Oxygen Saturation), HRV (Heart Rate Variability), and sleep stages. The device measures data at 10-second intervals, stores it in 5-minute batches, and transmits it to the server while recording movement data during sleep to analyze sleep efficiency.

Additionally, the device transmits data to the application via BLE communication and temporarily stores the data locally to prevent data loss.

The web server acts as an intermediary between the application and the backend API server, managing data flow and enhancing security.

The web server receives HTTP requests from the client and forwards them to the appropriate backend API server. It optimizes system performance by distributing requests across multiple backend API servers through load balancing.

The web server encrypts data using SSL/TLS encryption and protects the internal network with a reverse proxy. It routes requests to the appropriate server based on API endpoints (URL paths).

The backend API server processes client requests, executes business logic, and interacts with the database to store or retrieve data. It validates client request data and performs authentication and authorization checks based on JWT. The backend API server analyzes biometric data to generate user-specific results, such as stress indicators, HRV metrics, and sleep stage analysis. It downloads data uploaded to Cloud Storage, stores it in the database, generates analysis results, and returns the processed results to the client, optimizing the data analysis process.

The database serves as a central repository for storing and managing user biometric data and analysis results. It is based on MySQL and operates in a Cloud SQL environment. The database stores user IDs, biometric signal data (HR, HRV, EEG), analysis results, and stimulation setting data. It performs data insertion, retrieval, and updates through SQL queries and returns data based on client requests. The database maintains data integrity and ensures stability through regular automated backups and recovery systems.

Cloud Storage is a cloud-based storage system for storing large volumes of data uploaded from devices and smartwatches. It stores real-time received data in JSON format and maintains a data structure optimized for analysis and retrieval. Cloud Storage stacks data from devices and smartwatches and uploads it in 5-minute intervals. It manages data file indexes and metadata to support efficient data retrieval and analysis.

Devices and smartwatches transmit biometric data to the application via BLE, including ECG, EEG, actigraphy, and sound data. The application temporarily stores the data locally, stacks it in 5-minute intervals, and uploads it to Cloud Storage.

The application generates HTTP requests and sends them to the web server. The web server forwards the requests to the appropriate API server through load balancing. The API server inserts or retrieves data from the database and generates analysis results. The database returns the processed results to the API server, which refines them and sends them to the client. The processed results are transmitted to the application via the web server, allowing users to view them in real time.

This structure leverages BLE communication and Cloud Storage to receive and analyze data in real time.

According to an example embodiment, data security is enhanced, and data loss is prevented through the use of a reverse proxy in the web server and SSL/TLS encryption. The invention supports handling large-scale user requests with multiple API servers and database clustering. Additionally, the invention enables efficient data storage and retrieval through a JSON-based data structure and stack-based data management.

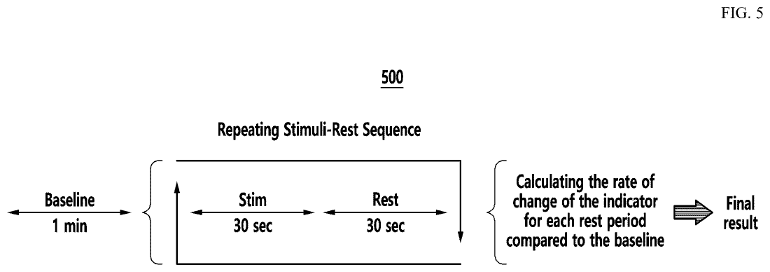

FIG. 5 illustrates the personalized stimuli setting algorithm 500.

Specifically, FIG. 5 provides a detailed step-by-step explanation of the entire process of the personalized stimuli setting algorithm according to the present invention, outlining the flow of baseline measurement, repetition of stimulation-rest sequences, calculation of indicator rate of changes for each interval, optimal stimuli setting, and final result delivery.

This algorithm is designed to perform real-time analysis based on biometric data received from users, determine the optimal stimulation settings, and provide results tailored to the user's physiological and psychological state.

Baseline measurement is the first step of the algorithm, aimed at assessing the user's initial condition and establishing a reference value for comparing changes in subsequent steps.

In this step, biometric data is received from the user for approximately one minute, and HRV and related metrics are calculated based on a stable state. Specifically, ECG (Electrocardiogram) is used to extract heart rate and heart rate variability, with key metrics including RMSSD, SDNN, and the LF/HF ratio.

The baseline quantitatively measures the user's current stable state and serves as a reference for calculating the rate of change between stimulation and rest, as well as for evaluating the significance of the stimulation effect.

The received data undergoes noise removal and normalization processes for analysis, during which the mean RMSSD value and LF/HF ratio are established as baseline values.

Once the baseline measurement is complete, the algorithm repeats a sequence of stimulation and rest, receiving user response data during each sequence.

The stimuli provided to the user are designed in various forms, such as binaural beats, electrical signals, or auditory stimuli, with each stimulation phase lasting 30 seconds. The rest period following the stimulation is also set to 30 seconds, during which the user's biometric data stabilizes, and the effects of the stimulation are evaluated.

The stimulation-rest sequence is performed according to a predefined number of repetitions, and the data received during each repetition is processed and analyzed in real time.

During the sequence, the received data is processed in real time and compared with the baseline values to evaluate the effects of the stimulation.

According to an example embodiment, HRV indicators such as RMSSD, SDNN, and the LF/HF ratio are calculated during each stimulation and rest period. Variability is assessed based on the R-R intervals in the ECG data to analyze stress levels and the state of the autonomic nervous system.

According to an example embodiment, the HRV indicators for each rest period are compared with the baseline values to calculate the rate of change. The rate of change is calculated by subtracting the baseline indicator from the rest period indicator, dividing the result by the baseline indicator, and multiplying by 100.

The rate of change may be calculated using [Equation 1].

$$Rate of Change = \frac{Rest Period Indicator - Baseline Indicator}{Baseline Indicator} \times 100 \qquad [ Equation 1 ]$$

For example, if the baseline SDNN value is 50 ms and it is measured as 60 ms during the rest period, the rate of change is calculated as 20%, as shown in [Equation 2]. The rate of change data calculated in each sequence is integrated and used to determine the optimal stimulation settings.

$$Rate of Change = \frac{60 - 50}{50} \times 100 = 20 \% \qquad [ Equation 2 ]$$

The analyzed data is used to quantitatively evaluate the effects of each stimulation option and serves as a basis for determining the optimal stimulation settings.

According to an example embodiment, a comprehensive score is calculated by assigning weights to each HRV indicator, with higher weights given to the LF/HF ratio when the goal is stress reduction.

The stimulation setting with the highest comprehensive score is predetermined as the optimal stimulation, which includes parameters such as the intensity, type, and duration of the stimulation.

The optimal stimulation settings are provided as personalized feedback to the user, including the optimized stimulation type, intensity, and duration. Users may view the results through the application interface, which visually represents the outcomes.

According to an example embodiment, the user's HRV rate of change, stress reduction level, and optimal stimulation settings are clearly communicated. For example, binaural beat stimulation may be provided at 15 Hz for 30 seconds, and electrical signal stimulation may be delivered at an intensity of 0.5 mA for 20 seconds.

This algorithm calculates the rate of change compared to the baseline through real-time data analysis and evaluates the user's response immediately.

According to an example embodiment, the reliability of results is ensured by basing them on scientifically validated data such as HRV indicators. The optimal stimulation is personalized based on each user's biometric data to maximize effectiveness.

This invention delivers accurate and reliable results through a systematic and step-by-step process, including baseline measurement, repetition of stimulation-rest sequences, calculation of indicator rate of changes, determination of optimal stimulation, and result delivery.

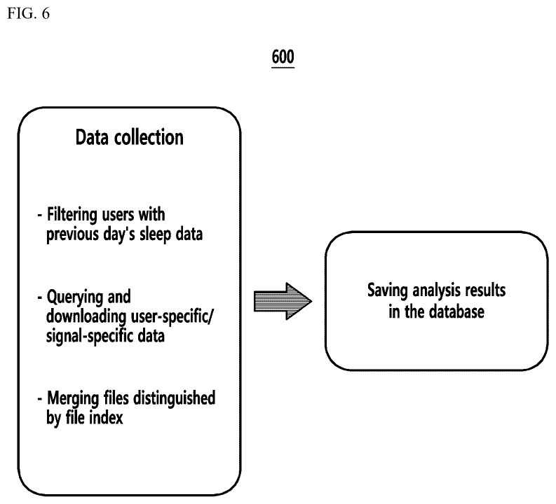

FIG. 6 illustrates sleep analysis through batch scheduling 600.

Specifically, FIG. 6 details the process consisting of the stages of data collection, signal-specific analysis, and storage of analysis results.

This system is designed to perform efficient and accurate processing from data collection to analysis and result storage through automated batch operations, aiming to provide personalized feedback and enable long-term sleep state management.

For example, batch operations are executed daily between 7 a.m. and 8 a.m., enhancing consistency in data processing and reliability in analysis.

Data collection is the initial stage of batch operations, involving filtering users who recorded sleep data from the previous night and downloading their data to prepare for analysis.

The analysis is limited to users who recorded sleep data the previous night. Filtering is conducted based on criteria such as whether data was recorded, storage status, and file integrity.

The filtered user list is registered in the batch operation queue and processed sequentially. User data is retrieved and downloaded from cloud platforms like Cloud Storage, including ECG, EEG, actigraphy, and sound data.

Data files are managed based on unique file indexes, preventing duplication, ensuring integrity, and facilitating the merging of signal data.

The downloaded data is merged by signal type and converted into a format suitable for analysis, ensuring consistency through synchronization processes. The data is inspected for damage or incompleteness, with damaged data excluded and subjected to a quality assurance process.

The received data is separated by signal type and processed through analysis methods tailored to each signal's characteristics.

ECG data is analyzed using libraries, undergoing noise removal and normalization processes, and HRV metrics (e.g., SDNN, RMSSD, pNN50, LF/HF ratio) are calculated. These results are used to evaluate the user's stress levels and analyze the state of their autonomic nervous system.

EEG data is analyzed using the Sleep ExpertNet AI model, which performs noise removal and normalization before classifying sleep stages into Wake, N1, N2, N3, and REM.

The analysis results are utilized to evaluate sleep quality and identify abnormal patterns. Actigraphy data is processed through a custom-developed analysis module to calculate WASO, total sleep time, and sleep efficiency, evaluating the correlation between user activity levels and sleep quality.

Sound data is segmented using libraries and classified into REM and non-REM stages using the dymn model, allowing for an assessment of the impact of environmental noise on sleep quality.

The analyzed data is organized by user and stored in the database. Storage items include HRV analysis results (e.g., SDNN, RMSSD, LF/HF ratio), EEG analysis results (sleep stage ratios and durations), Actigraphy analysis results (total sleep time, number of awakenings, sleep efficiency), and Sound analysis results (sound data for each sleep stage).

The data is organized by user ID, analysis date, and data type, enabling efficient retrieval and processing. The database schema is designed to optimize long-term analysis and pattern evaluation.

Data is regularly backed up, encrypted, and stored with access permissions restricted to maintain data integrity and security.

The system according to this invention executes batch operations at a designated time each day, automating the data analysis process. It processes large volumes of data in parallel to maximize processing speed and efficiency, providing accurate results using advanced analysis tools and AI models.

This system utilizes cloud-based infrastructure to provide scalability for handling data from multiple users simultaneously. It derives information to improve sleep quality through personalized feedback tailored to individual users.

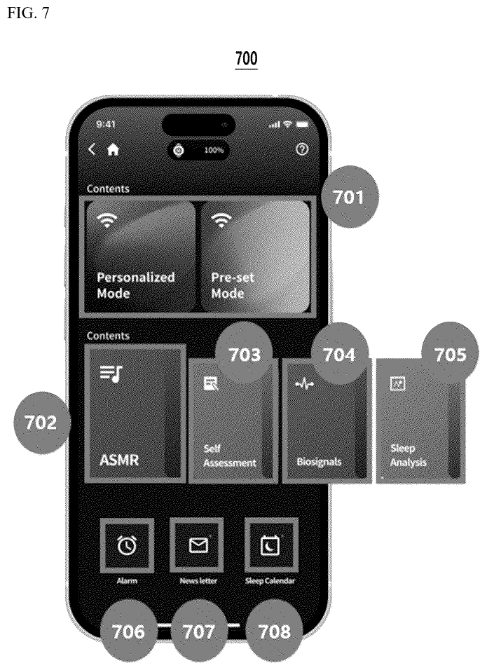

FIG. 7 illustrates the main content and user interface components of the application according to the present invention.

The application 700 is designed to improve users' health and sleep by offering features such as real-time analysis, feedback provision, stimulation settings, sleep analysis, and self-diagnosis based on users' biometric signal data. A user-friendly interface and intuitive functionality arrangement support convenient accessibility and efficient data utilization.

### Personalized Mode 701:

In Personalized Mode 701, the user's real-time biometric signal data is measured and analyzed to set personalized stimulation. Biometric data such as SpO2, HRV, and ECG is measured for approximately 2 minutes and transmitted to the server, where it is analyzed to generate a suitable stimulation recipe. Stimulation settings are automated, and users may freely select a stimulation duration of up to 40 minutes.

When stimulation begins, ASMR 702 with binaural beats is played, and users may activate a mute option if needed. This mode provides psychological stability to users in stressful states.

### Pre-Set Mode 701:

In Pre-set Mode 701, users may manually select and configure the type of stimulation. After selecting one of the provided stimulation recipes, users may freely choose a stimulation duration of up to 40 minutes.

When stimulation begins, ASMR 702 is played, and users may choose whether to mute the sound. Pre-configured stimulation may be used to achieve specific goals, such as promoting sleep.

### ASMR 702:

In ASMR 702, users may select and play ASMR content. The provided ASMR categories are divided into focus enhancement, psychological stability, and sleep induction. The content includes binaural beats, enhancing psychological stability and sleep induction effects.

Users may adjust the volume and playback speed according to their preferences, using the feature to alleviate stress or prepare for sleep.

### Self Assessment 703:

In Self Assessment 703, users may evaluate their sleep and psychological states. It allows users to assess sleep quality using PSQI, analyze insomnia levels through ISI, and measure anxiety and depression levels with BAI and BDJ. The survey results are scored and provided to the user, with recommendations for professional consultation if necessary. Users may periodically monitor their condition and develop a health management plan.

### Bio-Signals 704:

In bio-signals 704, users' biometric signal data is visualized in real time. It provides data such as PPG, actigraphy, sound, and SpO2 in graph form, updated in real time, enabling users to view data on an hourly basis. This feature allows users to monitor their condition in real time and understand their health status.

### Sleep Analysis 705:

In Sleep Analysis 705, the user's sleep state is analyzed based on the previous night's sleep data, and scored results are provided. Metrics such as total sleep time, sleep stage ratios, number of awakenings, and snoring sound data are analyzed to calculate a sleep score, which is visualized in graph form for the user. Users may analyze long-term sleep patterns and explore ways to improve them.

### Alarm 706:

In Alarm 706, users may set alarms for specific days of the week. Users may configure wake-up times and select alarm sounds, including those with binaural beats. The alarm is linked to the user's sleep analysis data and suggests optimal wake-up times, helping users maintain regular wake-up patterns and improve sleep efficiency.

### Newsletter 707:

In Newsletter 707, updates on device functionality, new feature introductions, and health management tips are provided to users. Information is delivered regularly through in-app notifications and emails, allowing users to stay updated on the latest features and health insights.

### Sleep Calendar 708:

In Sleep Calendar 708, users may review sleep scores and analysis results for specific dates and track changes in sleep patterns. Users may select a specific date from the calendar to view that day's sleep score, self-assessment results, and sleep stage ratios. Long-term data tracking provides a visualized report of score changes, enabling users to analyze their sleep state over time and set improvement goals.

The application 700 is designed with an intuitive layout and clear content arrangement, allowing users to access it easily.

It provides real-time analysis and feedback based on users' biometric data and delivers personalized stimulation settings and feedback tailored to each user's data and preferences.

Through features such as Self Assessment 703, Sleep Analysis 705, and ASMR 702 content, the application supports users' well-being and psychological stability, offering scientific and systematic assistance to help users accurately understand and improve their condition.

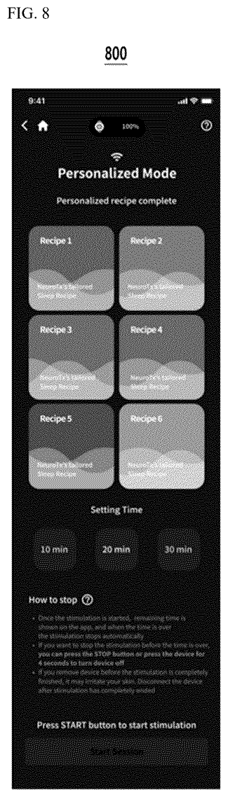

FIG. 8 illustrates the Personalized Mode for stimulation within the app service.

This mode includes functionality to provide optimized stimulation tailored to the user by setting personalized stimulation based on their biometric signal data. Users may easily select a stimulation recipe and set the stimulation duration through the application interface to receive customized stimulation.

First, as shown in FIG. 8, the user's real-time biometric data is measured. Signals such as SpO2, HRV, and ECG are measured for approximately 2 minutes and sent to the server for analysis.

The server selects a stimulation recipe appropriate for the user's current state based on the analysis results. The predetermined recipe is displayed on the application screen, and the user may choose one from the provided recipes.

Each recipe consists of specific stimulation waveforms and intensities designed to promote psychological stability and improve sleep.

The user receives stimulation for up to 40 minutes. Once stimulation begins, ASMR content containing binaural beats is automatically played, with the option to activate mute if needed. During the stimulation session, the application displays the remaining time on the screen, and users may stop the stimulation at any time before the set duration by pressing the “Stop” button.

At the top of the screen, the message “Personalized recipe complete” is displayed, indicating that stimulation customized for the user is being provided. The stimulation responds to the user's state and continues for up to 40 minutes. If the user is determined to be in a stable state, the stimulation may stop before the 40-minute mark. The maximum stimulation duration is 40 minutes.

At the bottom of the screen, instructional text is included, and the user may start the session by pressing the “Start Session” button.

This mode enhances the user experience by providing automated analysis and stimulation settings based on the user's biometric data. It also offers flexibility, allowing users to select stimulation aligned with their state and goals. The intuitive design of the interface ensures ease of use.

This Personalized Mode contributes to stress relief, sleep induction, and overall well-being improvement for users.

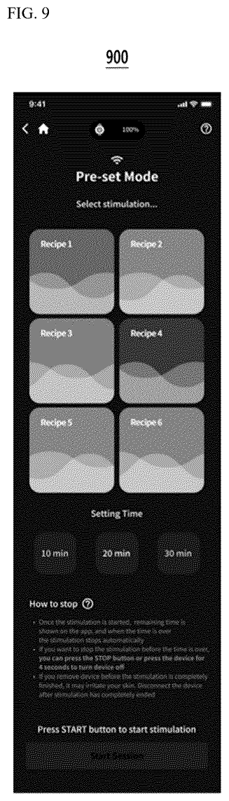

FIG. 9 provides a detailed explanation of the structure and operation of the Pre-set Mode in the application according to the present invention (900).

This mode is designed to allow users to directly select and execute one of the pre-provided stimulation options.

As illustrated in FIG. 9, the intuitive user interface enables users to easily choose the stimulation type and set the duration. Each option may be adjusted to suit the user's preferences and needs.

Pre-set Mode consists of three main processes: selecting a stimulation recipe, setting the duration, and starting the stimulation. Users may choose one of the options displayed on the screen, ranging from Recipe 1 to Recipe 8. Each recipe provides a stimulation pattern optimized for specific goals such as sleep induction, stress relief, focus enhancement, body relaxation, or dementia prevention, helping users select appropriate stimulation tailored to their objectives.

Users may autonomously select a duration of up to 40 minutes. The predetermined time serves as the criterion for automatically ending the stimulation session. Once the stimulation begins, ASMR containing binaural beats is automatically played, and users may activate the mute function if needed. This design aims to maximize psychological stability and relaxation during the stimulation session.

The bottom of the Pre-set Mode interface includes a feature allowing users to easily stop the stimulation session.

Users may press the Stop button during the session to immediately halt the stimulation or let the session continue until the set duration ends. This functionality is designed to enhance user convenience and ensure safety.

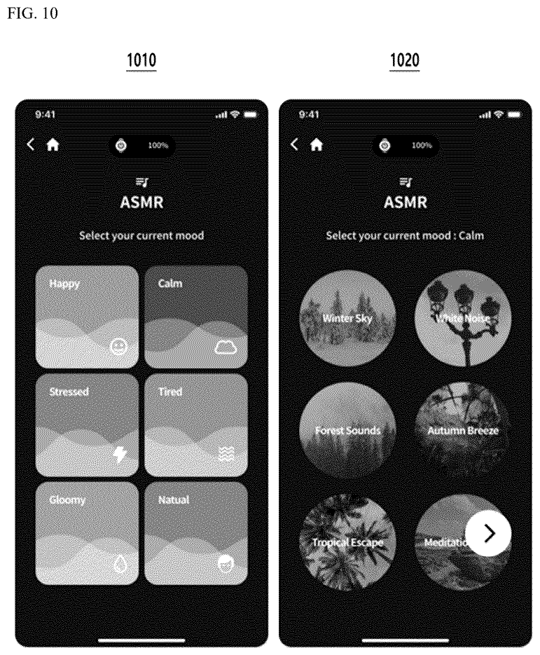

FIG. 10 illustrates the ASMR content available for playback within the app service.

The application provides ASMR content tailored to the user's emotional state, offering customized sound content, including binaural beats, to promote psychological stability and relaxation. Users may select and play content based on their current mood.

The application categorizes the user's current emotional state into groups such as “Happy,” “Calm,” “Stressed,” “Tired,” “Gloomy,” and “Natural” (reference number 1010). Users may select their emotional state from these categories, and a list of ASMR content suitable for the chosen state is displayed (reference number 1020).

For example, if the “Calm” state is predetermined, ASMR content such as “Winter Sky,” “White Noise,” “Forest Sounds,” “Autumn Breeze,” “Tropical Escape,” and “Meditation” is displayed. Users may select and play their preferred content from the list.

The ASMR content is designed to alleviate specific emotional states or provide psychological stability. Each piece of content includes binaural beats to enhance the user experience. Users may adjust the volume and playback speed during ASMR playback, allowing for a personalized sound environment.

The application provides an intuitive user interface, enabling users to easily navigate and select content.

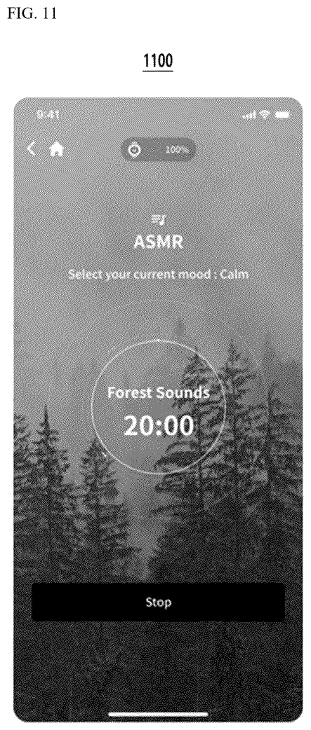

FIG. 11 illustrates the screen featuring binaural beats within the app service 1100.

According to FIG. 11, the ASMR content predetermined by the user is played alongside binaural beats, designed to provide optimized stimulation tailored to the user's state.

The screen in FIG. 11 is intuitively designed to enhance the user experience, allowing easy playback and control of ASMR content.

At the top of the screen, the predetermined mood (e.g., “Calm”) is displayed, determined by the emotional state chosen by the user on the previous screen. In the center, the name of the predetermined ASMR content (e.g., “Forest Sounds”) and the remaining playback time are displayed, enabling users to clearly understand the current content and the time left.

The center of the screen includes animated visual feedback that plays during ASMR content playback. This element enhances user immersion and intuitively conveys the playback progress.

The playback time decreases automatically based on the duration set by the user at the start of the session (e.g., 10 minutes, 20 minutes, 30 minutes), with the remaining time updated in real time on the screen.

At the bottom of the screen, a “Stop” button is placed, allowing users to stop playback at any time. Positioned for clarity and easy access, this button ensures usability. Pressing the button immediately stops the content, allowing users to select new content or navigate to other features of the application if desired.

This screen combines ASMR content with binaural beats to provide effects such as psychological stability, stress relief, and improved focus. Binaural beats contribute to enhancing the user's psychological state by delivering sound waves of specific frequencies to both ears.

The system is designed to maximize comfort and immersion during content playback, meeting individual needs and preferences through personalized features.

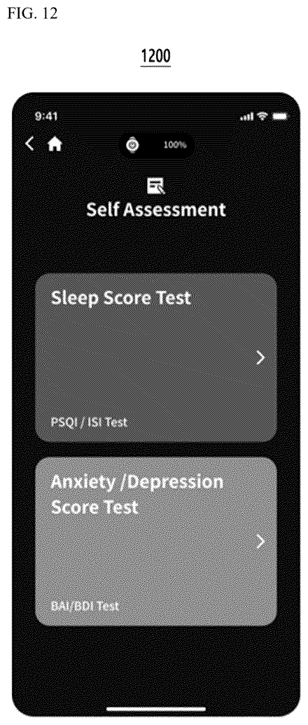

FIG. 12 illustrates the Self Assessment feature within the app service 1200.

Specifically, FIG. 12 presents an interface allowing users to self-assess their sleep and psychological health status.

The screen in FIG. 12 provides two primary evaluations: Sleep Score and Anxiety/Depression Score. Users may regularly check and manage their condition through these assessments.

The Self Assessment feature is divided into two main categories for user convenience. The first is the Sleep Score Test, designed to evaluate sleep quality, and the second is the Anxiety/Depression Score Test, aimed at assessing psychological health. Users may select and easily execute the desired test.

The Sleep Score Test is based on the Pittsburgh Sleep Quality Index (PSQI) and the Insomnia Severity Index (ISI).

The PSQI consists of 10 questions evaluating the user's sleep quality. Results range from 0 to 21 points and are categorized as Good Sleep Quality or Bad Sleep Quality.

The ISI consists of 7 questions assessing the severity of insomnia. Results range from 0 to 28 points and are classified into four categories: No Clinically Significant Insomnia, Subthreshold Insomnia, Moderate Insomnia, and Severe Clinical Insomnia.

The Anxiety/Depression Score Test is based on the Beck Anxiety Inventory (BAI) and the Beck Depression Inventory (BDI).

The BAI consists of 21 questions measuring anxiety levels. Results are categorized as Low Anxiety, Moderate Anxiety, and Potentially Concerning Levels of Anxiety.

The BDI evaluates depression severity through 21 questions, providing results categorized into six levels: Normal, Mild Mood Disturbance, Borderline Clinical Depression, Moderate Depression, Severe Depression, and Extreme Depression.

Each test features a simple and intuitive user interface to optimize the user experience. Users may view their scored results in real time upon completing the test and, if necessary, request professional consultation or additional support.

This feature serves as a tool to help users systematically manage their condition. By providing reliable data and supporting regular self-assessment, it aims to improve users' health and manage their status effectively.

FIG. 12 clearly explains the evaluation process and the method of delivering results.

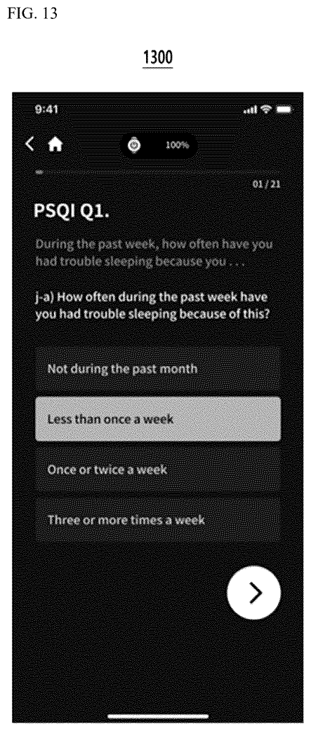

FIG. 13 illustrates the Sleep Quality Index (PSQI) within the app service 1300.

FIG. 13 provides a detailed explanation of the interface designed to allow users to evaluate their sleep quality through self-assessment.

The Pittsburgh Sleep Quality Index (PSQI) is a widely used sleep evaluation tool in neurology, designed to assess various aspects of sleep quality and measure the presence and severity of sleep problems.

The questionnaire consists of 10 questions, each receiving information about the user's sleep experience over the past month. Topics include sleep onset time, frequency of awakenings, total sleep time, and overall sleep quality. Each question is presented with multiple-choice answers, and the user progresses to the next question automatically after making a selection.

The questions are structured to be user-friendly, with responses based on the frequency of sleep-related issues over the past month. The four response options are:
“Not during the past month”
“Less than once a week”
“Once or twice a week”
“Three or more times a week”

Upon completing the questionnaire, the responses are scored to generate a PSQI total score. The score ranges from 0 to 21, with higher scores indicating lower sleep quality. Scores are categorized as follows:
0-5 points: Good sleep quality
6 points or above: Indicates the presence of sleep quality issues

After completing the questionnaire, users may view their PSQI score, which is stored in the app's database. The score is displayed visually and may be compared with past scores to track changes in sleep patterns.

If the PSQI score exceeds the threshold, the app may recommend that the user consider professional consultation.

The interface depicted in FIG. 13 is designed to be simple and intuitive, enabling users to easily understand and answer the questions. Each question and its response options are clearly distinguished, and users may respond with a simple tap.

A progress bar indicates the completion status, and the current question number (e.g., 1/10) is displayed at the top of the screen.

PSQI is a validated tool in the fields of neurology and sleep research, enabling users to reliably evaluate their sleep quality. The simple and intuitive interface is designed for ease of use across all age groups.

User scores are stored for long-term analysis, allowing for the identification of changes in sleep patterns and the development of improvement strategies. The PSQI results help in the early identification of sleep issues and encourage users to seek professional advice or take remedial actions.

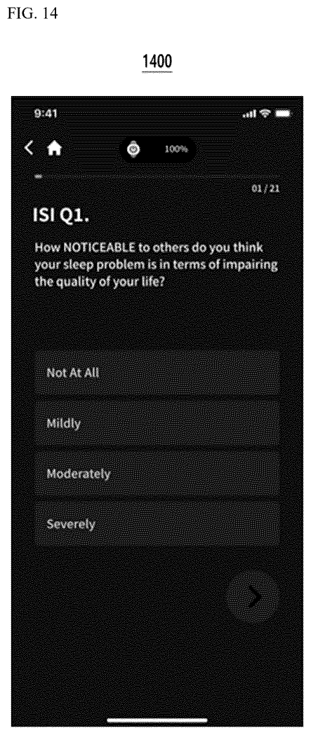

FIG. 14 illustrates the Insomnia Severity Index (ISI) within the app service 1400.

FIG. 14 provides a detailed explanation of the interface designed to allow users to self-assess the severity of their insomnia.

The ISI (Insomnia Severity Index) is a widely used tool in neurology and psychology to evaluate the severity of insomnia. This application integrates ISI for convenient user access and usage.

The questionnaire consists of 7 questions, each receiving information to assess symptoms and impacts related to the user's sleep problems. The questions cover topics such as:
The degree of difficulty falling asleep
The degree of difficulty staying asleep
The frequency and severity of early awakening
The discomfort caused by sleep problems
The impact of sleep problems on the user's quality of life
The level of confidence in managing insomnia issues

This implementation makes the ISI accessible for users to evaluate their sleep conditions effectively.

Each question is presented in a multiple-choice format for ease of response, offering four answer options: Not at all, Mildly, Moderately, and Severely.

Upon completing the questionnaire, each response is scored to calculate the total ISI score. Scores range from 0 to 28, with higher scores indicating greater severity of insomnia. The scores are categorized as follows:
0-7 points: No clinically significant insomnia
8-14 points: Subthreshold insomnia
15-21 points: Moderate clinical insomnia
22-28 points: Severe clinical insomnia

After completing the questionnaire, users may view their ISI score, which is stored in the app's database. Scores are visually displayed, allowing users to compare their current score with previous scores to track changes in sleep patterns and insomnia severity. Users with high scores may be advised through the app to consider professional consultation.

The interface depicted in FIG. 14 enables users to complete the questionnaire easily. Each question and response option is clearly distinguished, and users may respond with a simple touch input. At the top of the screen, the current question number (e.g., Q1/7) and a progress bar are displayed, providing users with a clear indication of their progress.

The ISI is a scientifically validated tool for quantitatively assessing the severity of insomnia. Its user-friendly and simple interface is designed for accessibility across all age groups.

Through long-term data storage and tracking, the app allows for analysis of changes in a user's insomnia condition and the development of strategies for improvement. The ISI results contribute to the early detection of insomnia issues and encourage users to seek professional consultation or take remedial actions, ultimately improving their quality of life.

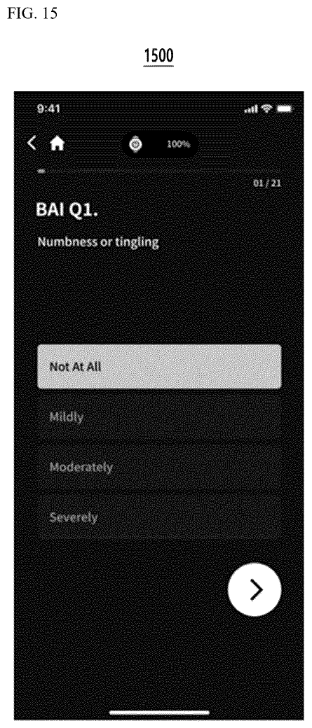

FIG. 15 is a diagram (1500) visually illustrating the process of measuring the Beck Anxiety Inventory (BAI) in the application according to the present invention. It provides a detailed explanation of the interface designed to allow users to self-assess their anxiety levels.

The BAI is a widely used tool in psychology and psychiatry for evaluating the severity of anxiety symptoms. This application integrates the BAI in a user-friendly and intuitive format to enhance accessibility.

The BAI consists of 21 questions, each designed to receive information related to physical and cognitive anxiety symptoms. The question items include physical symptoms (e.g., tingling in hands and feet, heart palpitations) and cognitive symptoms (e.g., fear, difficulty concentrating). Users respond based on how frequently they experienced these symptoms in the past week. Each question offers four response options: “Not at all,” “Mildly,” “Moderately,” and “Severely,” which are converted into scores.

Upon completing the questionnaire, all responses are scored, and the total score ranges from 0 to 63. The scores are categorized as follows:
0-9 points: Low anxiety
10-18 points: Moderate anxiety
19 points or higher: Potentially concerning levels of anxiety

These scores quantitatively evaluate the user's anxiety level and may be used to recommend professional consultation if necessary.

After completing the questionnaire, users may instantly view their BAI score. This score is stored in the application's database, allowing for long-term tracking of changes in their condition. The score is displayed as a visual graph, enabling users to compare their results with previous evaluations and monitor improvements or deteriorations in their anxiety state.

The interface depicted in FIG. 15 ensures that questions and response options are clearly distinguished, allowing users to respond easily via touch input. At the top, the current question number and a progress bar are displayed, providing users with a clear indication of their progress through the questionnaire.

The BAI is a scientifically validated tool for anxiety assessment, enabling users to evaluate their condition reliably and take the initial steps toward anxiety improvement. Users may quantitatively analyze their mental state using this tool, making informed decisions regarding health management and consultation. The application's BAI functionality supports users in managing and improving their anxiety effectively.

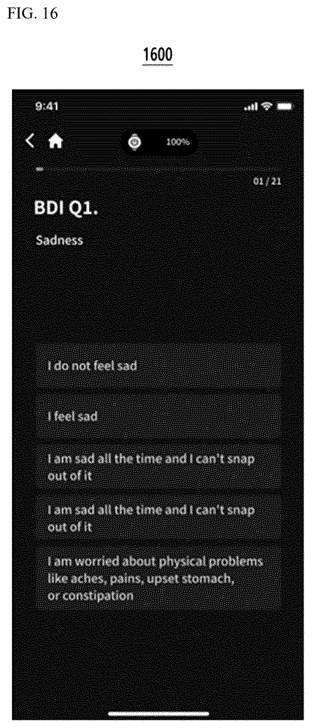

FIG. 16 is a diagram (1600) visually illustrating the process of measuring the Beck Depression Inventory (BDI) in the application according to the present invention. FIG. 16 provides a detailed explanation of the interface designed to allow users to self-assess their level of depression.

The BDI is a widely used tool in psychology and psychiatry to evaluate the severity of depression. The application implements this tool in a user-friendly manner for ease of use.

The depression assessment process consists of 21 questions, each designed to evaluate various aspects of depression, including emotional, behavioral, and physical symptoms. Users assess their condition by selecting answers to the questions.

Each question offers four multiple-choice options, including the following:
“I do not feel sad”
“I feel sad”
“I am sad all the time and I can't snap out of it”
“I am so sad or unhappy that I can't stand it”

Once a response is predetermined, it is scored and contributes to the total BDI score.

The BDI score ranges from 0 to 63, with higher scores indicating more severe depression. The scores are categorized as follows:
0-13 points: Normal
14-19 points: Mild mood disturbance
20-28 points: Borderline clinical depression
29-63 points: Severe depression

After completing the assessment, users may view their BDI score, which is stored in the application's database. The score is visualized in a graph, allowing users to track changes in their depression levels over time by comparing current results with previous ones. If the depression score is high, the application recommends professional consultation.

The interface depicted in FIG. 16 clearly distinguishes each question and response option. Users may select answers easily via touch input. At the top of the interface, the current question number (e.g., Q1/21) and a progress bar are displayed, providing users with a clear indication of their progress through the assessment.

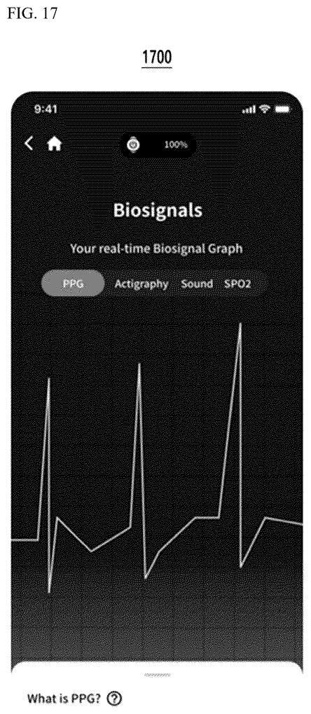

FIG. 17 is a diagram (1700) visually illustrating the bio-signals feature in the application according to the present invention, which allows users to monitor biometric signal data in real time and provides instant insights into their condition.

The bio-signals feature delivers real-time data on PPG (Photoplethysmography), Actigraphy (Physical Activity), Sound (Acoustic Data), and SPO2 (Oxygen Saturation), offering users intuitive information about their health status.

On the bio-signals page, data for PPG, Actigraphy, Sound, and SPO2 are organized into separate tabs. Users may select any tab to view the desired biometric signal data.

The graph displayed in FIG. 17 uses PPG data as an example, visualizing the user's heart rate variability pattern in real time. Each dataset is updated in time intervals, enabling users to monitor changes in their condition immediately through continuous updates.

This feature provides key indicators related to personalized health management in real time. For example:

PPG data reflects heart rate variability and may be used to assess stress levels or exercise states.

Actigraphy data captures physical activity and sleep patterns.

Sound data analyzes the impact of environmental noise during sleep.

SPO₂ data monitors blood oxygen levels, offering insights into respiratory health.

The interface is designed to ensure easy access to the data. At the top, the currently predetermined data type (PPG, Actigraphy, Sound, SPO₂) is displayed, while a brief explanation of the predetermined data type is provided below the graph.

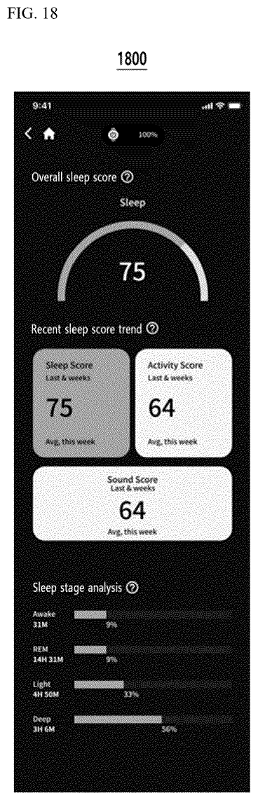

FIG. 18 is a diagram 1800 visually illustrating the Sleep Analysis feature in the application according to the present invention. This feature analyzes the user's sleep state and provides a quantified score based on the analysis.

FIG. 18 is designed to evaluate the user's overall sleep condition comprehensively and suggest areas for improvement. Alongside an overall sleep score, Sleep Analysis provides detailed insights into the user's sleep patterns.

At the top of FIG. 18, the overall sleep score is displayed out of 100 points, representing a quantified measure of the user's overall sleep quality. Below the overall sleep score, a graph shows trends in sleep scores over the past six weeks, accompanied by Sleep Score, Activity Score, and Sound Score metrics. Users may compare each score with the weekly average to track changes in their sleep patterns.

At the bottom of FIG. 18, detailed analysis results of the user's sleep stages are presented. These are categorized into four stages: Awake, REM (Rapid Eye Movement), Light Sleep, and Deep Sleep. The duration and percentage of each stage are displayed in a graphical format, allowing users to intuitively understand their sleep states.

The sleep analysis results are generated based on key biometric data: ECG data is used to assess sleep quality through heart rate variability indicators.

EEG data is analyzed using AI-powered sleep stage analysis models to determine the distribution of Wake, N1, N2, N3, and REM stages.

Actigraphy data evaluates sleep efficiency, total sleep time, and major rest periods by analyzing the user's physical activity patterns.

Sound data analyzes audio recorded during sleep to evaluate sleep stage distribution and environmental factors.

The Sleep Analysis feature enables users to scientifically understand their sleep patterns and take actionable steps to improve their sleep quality.

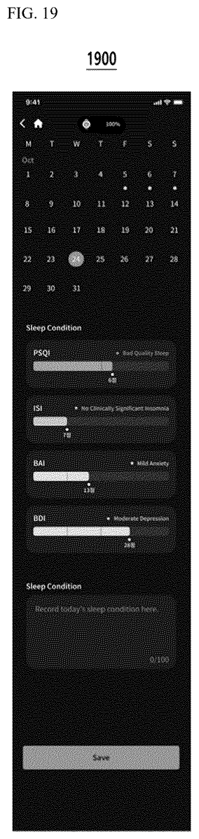

FIG. 19 is a diagram illustrating the Sleep Calendar feature within the application service. It visually represents the functionality that allows users to view their sleep records and self-assessment results in a calendar format, enabling them to track their sleep and psychological state at a glance.

The Sleep Calendar is designed to display a monthly calendar where users may select a specific date to view their sleep score and self-assessment results for that day. Users may visually check their scores for PSQI (Pittsburgh Sleep Quality Index), ISI (Insomnia Severity Index), BAI (Beck Anxiety Inventory), and BDI (Beck Depression Inventory) for the predetermined date. Each score is accompanied by a status descriptor (e.g., Bad Quality Sleep, No Clinically Significant Insomnia) to help users intuitively understand their condition.

At the bottom of the interface, a text input field is provided where users may record additional notes about their sleep condition. Users may enter supplementary information related to their sleep and save it by clicking the save button. This feature supports users in managing and analyzing their sleep and psychological state in greater detail.

The Sleep Calendar generates a monthly report based on historical data, visually presenting trends in score changes. These reports help users clearly identify patterns in their sleep and psychological state, enabling them to set improvement goals.

In FIG. 19, clicking on a specific date in the calendar instantly displays the relevant data. Each score is accompanied by a brief explanation and visual indicators to enhance user understanding. This feature enables users to comprehensively manage their sleep and psychological state and set clear directions for improvement.

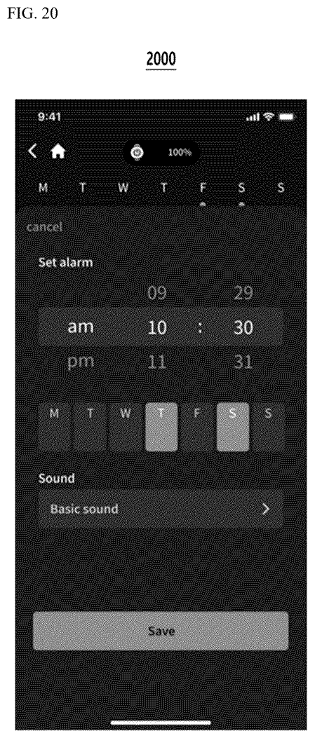

FIG. 20 is a diagram illustrating the Alarm feature within the application service 2000. More specifically, FIG. 20 visually represents the Alarm function in the application according to the present invention, which allows users to set and manage alarms easily to support sleep management and a regular lifestyle.

The Alarm function provides an interface for setting alarm times by day of the week. Users may select the desired time and day to set an alarm. The configured alarms are visually displayed on the screen. Users may activate or deactivate alarms for specific days and choose between default sounds or binaural beats as the alarm tone, aiding in sleep management and optimizing wake-up patterns.

At the top of the interface, the alarm time is displayed with an intuitive AM/PM selection for easy configuration. The day selection area is presented as rectangular buttons, with predetermined days visually highlighted for clarity. At the bottom, a dropdown menu allows users to select the alarm sound based on their preferences. After completing all settings, users may press the “Save” button to save the alarm configuration.

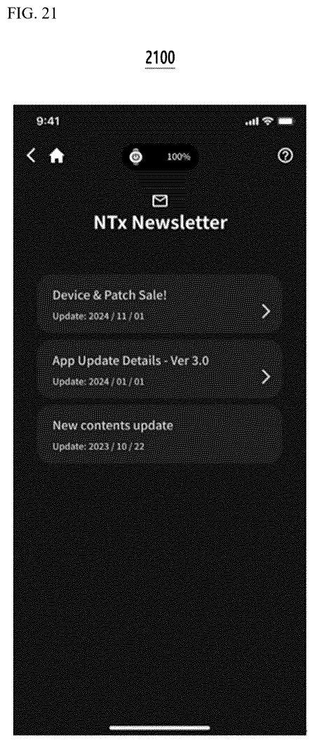

FIG. 21 is a diagram illustrating the Newsletter feature within the application service 2100.

More specifically, FIG. 21 visually represents the Newsletter function in the application according to the present invention, which allows users to receive information such as device updates, new feature introductions, and additional content updates. This feature is designed to enable users to quickly and easily stay informed about the latest updates.

The Newsletter screen provides a list format of items related to the latest updates. Each item includes a title and the update date, allowing users to easily review update information in chronological order. For instance, items like “Device & Patch Sale!”, “App Update Details—Ver 3.0”, “New Contents Update” are displayed, and clicking on an item redirects the user to a detailed page with related information.

At the top of the screen, the section title “NTx Newsletter” is prominently displayed, helping users immediately understand the purpose of the current screen. Each item is designed as a rectangular button, enabling intuitive access in a touch-based interface. The date displayed below each item indicates the recency of the update, helping users quickly identify the most recent information.

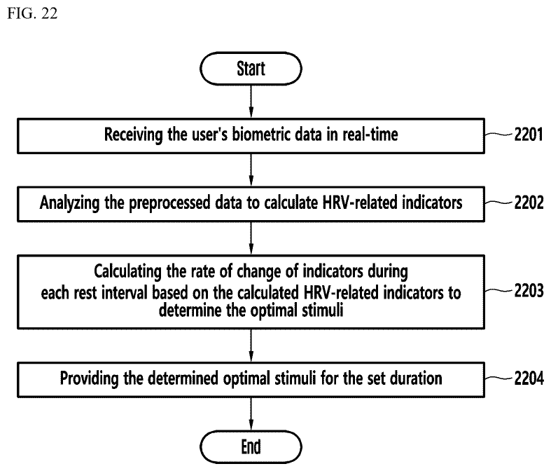

FIG. 22 illustrates the operation method of the system for providing personalized stimuli according to an exemplary embodiment.

FIG. 22 visually represents the entire process of receiving and analyzing biometric signal data, determining appropriate stimuli based on the user's condition, and delivering the stimuli. The user's biometric signal data is received in real-time through wearable devices or sensors, capturing various types of signals.

As shown in step 2201, the system receives the user's biometric signal data in real time. ECG measures heart rate and heart rate variability (HRV) to assess stress and relaxation states. EEG analyzes brainwave data to evaluate sleep stages and sleep quality. SPO₂ monitors blood oxygen saturation to analyze respiratory conditions. Actigraphy receives physical activity data to track movement and sleep patterns. Sound data captures environmental noise to assess factors affecting sleep quality. The received data is transmitted to the server via Bluetooth Low Energy (BLE) protocol, encrypted to enhance security during transmission.

As shown in step 2202, the preprocessed data is analyzed to calculate HRV-related metrics. During this stage, the received biometric signal data undergoes preprocessing. In step (2202), noise is removed from raw data, and filtering techniques are applied to eliminate measurement errors and environmental interference. The data is normalized to enhance comparability and sampled at consistent time intervals. From the heart rate data, NN intervals are extracted, and HRV metrics such as SDNN, RMSSD, LF/HF ratio, and PNN50 are calculated to analyze heart rate variability. This data accurately reflects the user's psychological and physiological state.

In step 2203, the system calculates the rate of changes of HRV-related metrics during each rest interval to determine the optimal stimuli. Specifically, in step 2203, the preprocessed data is analyzed to compute HRV rate of changes, which are then used to evaluate the user's condition. A decrease in HRV metrics indicates a stressed state, while an increase signifies psychological stability. A low LF/HF ratio may suggest physical fatigue. Based on the analysis, the system selects the most suitable stimuli from various types, such as binaural beats, ASMR content, and other frequency-based stimuli. The user's preferences and historical data are considered when configuring the stimuli, and the stimuli duration is automatically adjusted according to the user's specified time.

As shown in step 2204, the determined stimuli is delivered for the user's set duration. Binaural beats and ASMR content are played through audio devices, with frequency and volume adjusted according to the user's condition. During the stimuli provision, real-time monitoring of biometric signal data is performed. If the stimuli effect is insufficient, the intensity or type of stimuli is automatically adjusted. Once the set duration ends, the stimuli is terminated, and the final biometric data is stored on the server for further analysis.

Real-time data visualization allows users to view changes in their HRV metrics through graphs. The system's long-term data storage feature enables tracking of sleep patterns, stress management, and psychological state changes. A simple and intuitive interface ensures that anyone may easily use the system.

Ultimately, by utilizing the present invention, users may monitor their biometric signal data in real-time and intuitively understand their condition. The system provides personalized stimuli based on biometric indicators such as HRV, enhancing stress relief and psychological stability. Additionally, the invention improves the accuracy of biometric data analysis through noise removal and data normalization and learns from historical data to deliver stimuli that reflect the user's preferences and condition, thereby continuously improving the user experience. Furthermore, personalized stimuli settings improve sleep quality and support the formation of regular sleep patterns.

The above-described device may be implemented using hardware components, software components, and/or a combination of hardware and software components. For example, the devices and components described in the embodiments may be implemented using one or more general-purpose or special-purpose computers, such as a processor, controller, ALU (arithmetic logic unit), digital signal processor, microcomputer, FPGA (field programmable gate array), PLU (programmable logic unit), microprocessor, or any other device capable of executing and responding to instructions.

The processing device may execute an operating system (OS) and one or more software applications running on the OS. Additionally, the processing device may access, store, manipulate, process, and generate data in response to software execution. For simplicity of explanation, a single processing device is described in some cases; however, those skilled in the art will understand that the processing device may include multiple processing elements and/or various types of processing elements. For example, the processing device may include multiple processors or a combination of a processor and a controller. Other processing configurations, such as parallel processors, are also possible.

Software may include computer programs, code, instructions, or any combination thereof, and may configure the processing device to operate as desired or instruct the processing device independently or collectively. Software and/or data may be embodied permanently or temporarily in any type of machine, component, physical device, virtual equipment, computer storage medium or device, or transmitted signal wave to be interpreted or processed by the processing device or to provide instructions or data to the processing device. Software may be distributed across networked computer systems, stored, or executed in a distributed manner. Software and data may also be stored on one or more computer-readable recording media.

Methods according to embodiments may be implemented as program instructions executed through various computer means and recorded on computer-readable media. Such computer-readable media may include program instructions, data files, and data structures either alone or in combination. Program instructions recorded on the medium may be specifically designed and configured for the embodiments or may be available and used by those skilled in the field of computer software.

Examples of computer-readable recording media include magnetic media such as hard disks, floppy disks, and magnetic tapes; optical media such as CD-ROMs and DVDs; magneto-optical media such as floptical disks; and hardware devices configured to store and execute program instructions, such as ROM, RAM, and flash memory. Examples of program instructions include machine code generated by compilers and high-level language code executable by a computer using interpreters or other tools. The above-described hardware devices may be configured to operate as one or more software modules to perform operations of the embodiments, and vice versa.

Although embodiments have been described with reference to specific figures, those skilled in the art will understand that various modifications and variations are possible based on the disclosure. For example, described technologies may be performed in a different order than described, or components of the systems, structures, devices, and circuits may be combined or integrated in different ways, replaced, or substituted with other components or equivalents, achieving appropriate results.

Therefore, other implementations, other embodiments, and equivalents that fall within the scope of the claims are also included in the scope of the following claims.

What is claimed is:

1. A system for providing personalized stimuli comprising:
a data receiving unit configured to receive a user's biometric signal data in real time;
a data preprocessing unit configured to preprocess the received biometric signal data by removing noise and normalizing the data;
a data analyzing unit configured to analyze the preprocessed biometric signal data to calculate an HRV (Heart Rate Variability) index;
a stimulation determining unit configured to determine an optimal stimulation by calculating at least one rate of change of the HRV index for resting intervals based on the calculated HRV index; and
a stimulation providing unit configured to provide the determined optimal stimulation for a predetermined period of time.

2. The system of claim 1,
wherein the data receiving unit is configured to stack data on a local device in predetermined time units through short-range wireless communication and to upload the data to a cloud storage.

3. The system of claim 1,
wherein the data receiving unit is configured to store biometric signal data in a structured JSON (JavaScript Object Notation) format along with metadata including user ID, experiment ID, timestamp, and data file index.

4. The system of claim 1,
wherein the data preprocessing unit is configured to perform noise removal and normalization on the received biometric signal data, and to segment voice data using a library.

5. The system of claim 1,
wherein the data analyzing unit is configured to calculate WASO (Wake After Sleep Onset), total sleep time, major rest periods, and sleep onset latency based on actigraphy data.

6. The system of claim 1,
wherein the data analyzing unit is configured to calculate a user-specific sleep score by integrating HRV indicators, EEG sleep stage distributions, and actigraphy indicators based on the user's sleep data.

7. The system of claim 1,
wherein the data analyzing unit is configured to classify sleep stages from the received biometric signal data using a pre-trained artificial intelligence model.

8. The system of claim 1,
wherein the stimulation determining unit is configured to calculate the rate of change in the HRV index for each resting interval relative to a baseline index and to compute a comprehensive score by applying weights to each index.

9. The system of claim 1,
wherein the stimulation determining unit is configured to select the stimulation setting with the highest comprehensive score based on the optimal rate of change in indicators for each stimulation setting option.

10. The system of claim 1,
wherein the stimulation providing unit is configured to provide stimulation including binaural beats for the duration of the predetermined stimulation setting.

11. A method of operating a system for providing personalized stimuli, comprising:
receiving a user's biometric signal data in real time;
preprocessing the received biometric signal data by removing noise and normalizing the data;
analyzing the preprocessed data to calculate an HRV (Heart Rate Variability) index;
determining an optimal stimulation by calculating a rate of change of the HRV index for each resting interval based on the calculated HRV index; and
providing the determined optimal stimulation for a predetermined period of time.

12. The method of claim 11,
wherein the step of receiving the biometric signal data in real time comprises:
stacking the biometric signal data on a local device in predetermined time units via short-range wireless communication and uploading the data to cloud storage.

13. The method of claim 11,
wherein the step of determining an optimal stimulation by calculating a rate of change of the HRV index for each resting interval based on the calculated HRV index comprises:
calculating the rate of change of the HRV index for each resting interval relative to a baseline index, and calculating a comprehensive score by applying weights to the respective indices.

14. The method of claim 11,
wherein the step of determining an optimal stimulation by calculating a rate of change of the HRV index for each resting interval based on the calculated HRV index comprises:
selecting the stimulation setting with the highest comprehensive score based on the optimal rate of change of the index for each stimulation setting option.

15. A non-transitory computer-readable storage medium having programmable instructions stored therein, that when executed by one or more hardware processors, cause the one or more hardware processors to:
receiving a user's biometric signal data in real time;
preprocessing the received biometric signal data to remove noise and normalize the data;
analyzing the preprocessed data to calculate an HRV (Heart Rate Variability) index;
determining an optimal stimulation by calculating a rate of change of the HRV index for each resting interval based on the calculated HRV index; and
providing the determined optimal stimulation for a predetermined period of time.
